(19) United States
(12) Patent Application Publication
(10) Pub. No.: US 2006/0275504 A1
(43) Pub. Date: Dec. 7, 2006

(54) Methods and compositions for augmenting cancer chemotherapeutic agents

(75) Inventor: Ming-Feng Chen, Changhua (TW)

Correspondence Address: LADAS & PARRY, 26 WEST 61ST STREET, NEW YORK, NY, 10023, US

(73) Assignee: TTY BIOPHARM COMPANY LIMITED

(21) Appl. No.: 11/146,759

(22) Filed: Jun. 7, 2005

(51) Int. Cl. A61K 33/36; A61K 33/24; A61K 31/704; A61K 31/12; A61K 31/375; A61K 31/7072

(52) U.S. Cl. 424/623; 514/34; 514/682; 514/474; 424/649; 514/49

(57) ABSTRACT

This invention provides methods and pharmaceutical compositions/therapy combination product for cancer treatment and/or augmenting the effectiveness of cancer chemotherapeutic agents by use of a combination of ascorbic acid and vitamin K3.

### FIELD OF THE INVENTION

This invention relates to methods and compositions for augmenting the effectiveness of cancer chemotherapeutic agents, particularly relates to the use of a combination of ascorbic acid and vitamin K3 for augmenting the effectiveness of cancer chemotherapeutic agents.

### DESCRIPTION OF THE PRIOR ART

Tumors/cancers have always been a threat to the health of human beings. For many years, the medicinal field has endeavored to research and develop effective medicines for the treatment of tumors/cancers. In many cases, surgical intervention may be effective for tumor/cancer treatment. Moreover, a common treatment for malignant tumors is the administration of suitable chemotherapeutic agents, i.e., chemotherapy. Chemotherapy is based on the use of drugs that are selectively toxic (cytotoxic) to cancer cells. Several general classes of chemotherapeutic drugs have been developed, including drugs that interfere with nucleic acid synthesis, protein synthesis, and other vital metabolic processes.

Early in the 20^{th} century, arsenic compounds were used to treat chronic myelogenous leukemia and malignant lymphoma. Recently, arsenic trioxide (As₂O₃) has been used in the treatment of de novo and refractory acute promyelocytic leukemia (APL) patients, with high rates of response and hematological/molecular remission achieved (Soignet S L, et al., N Engl J Med 1998; 339:1341-1348). Clinical response has also been obtained for patients with human T cell lymphotropic virus type I associated adult T cell leukemia/lymphoma (Bazarbachi A. & Hermine O., Virus Res., 2001; 78:79-92). Further, As₂O₃ inhibits cell growth and induces apoptosis in certain types of cancer, including APL (Shao W. et al., J Natl Cancer I 1998; 90:124-133), hepatoma (Siu K P, et al., Life Sci. 2002; 71:275-285) as well as pancreatic (Li X, et al., Anticancer Res 2002; 22:2205-2213) and gastric (Jiang X H, et al., Init J Cancer 2001; 91:173-179) carcinomas.

It was reported that when arsenic trioxide was administered intravenously at a dose of 10 mg/d to patients with relapsed acute promyelocytic leukemia, a complete remission (CR) rate of 90% could be achieved (Shen Z. X. et al., Blood (1997), 89:3354). It was also reported that, other than arsenic trioxide, arsenic-containing substances such as “Composite Indigo Naturalis Tablets” containing arsenic sulfide (As₂S₂) and pure tetraarsenic tetraslfide (As₄S₄) can achieve complete remission rates of 98% and 84.9%, respectively (Wang Z. Y., Cancer Chemother Pharmacol (2001), 48 (suppl 1):S72-S76).

In 2001, the National Institutes of Health (NIH) of the United States proceeded with clinical trials of arsenic trioxide in hematologic and solid tumors, and indicated in their reports that arsenic trioxide can inhibit growth of many cancer cell lines, and promote apoptosis in the cancer cell lines. The clinical trials of arsenic trioxide conducted in connection with hematologic malignancies include, in addition to acute promyelocytic leukemia, acute myeloid leukemia (AML), acute lymphocytic leukemia, chronic myelogenous leukemia (CML), non-Hodgkin's lymphoma, Hodgkin's lymphoma, chronic lymphocytic leukemia, myelodysplastic syndrome and multiple myeloma. The clinical trials of arsenic trioxide conducted in connection with solid tumors include prostate cancer, cervical cancer and bladder cancer (Murgo A. J., The Oncologist (2001), 6 (suppl 2):22-28).

In September 2000, the Food and Drug Administration (FDA) of the United States approved arsenic trioxide as an orphan drug for treating acute promyelocytic leukemia. The Department of Health (DOH) of Taiwan, R.O.C. approved marketing of an arsenic trioxide-containing pharmaceutical preparation (“ASADIN Injection”; License No. 000005) by TTY Biopharm Co., Ltd. (Taiwan), in January 2002.

5-FU has been another one of the most famous chemotherapy drugs for treating many cancers for decades; it binds to an enzyme inside of the cancer cells and thereby exerts its anticancer effect on the cells.

Adriamycin (generally known as doxorubicin) is a common chemotherapy agent in the treatment of Hodgkin's disease and Nonhodgkin's lymphoma. It is more often than not used in conjunction with other chemotherapy agents in a multidrug regimen. It is a cytotoxic anthracycline antibiotic isolated from cultures of Streptomyces peucetius var. caesius. As such, it is an antineoplastic-adriamycin which interferes with the growth of cancer cells. It is one of the older chemotherapy drugs which have been used for decades for several types of cancer.

Cisplatin (sis-PLA-tin) belongs to the group of medicines known as alkylating agents. It is used to treat cancer of the bladder, ovaries and testicles. It may be used to treat other types of cancer. Cisplatin interferes with the growth of cancer cells, which are eventually destroyed.

Ascorbic acid, also known as Vitamin C, a water-soluble vitamin, is important in forming collagen that gives structure to bones, cartilage, muscle and blood vessels. It is known that ascorbic acid can be used as an alternative chemotherapy or as an adjuvant to lessen side effects associated with it. For example, ascorbic acid is claimed to improve the anti-neoplastic activity of doxorubicin (DOX), cisplatin (DDP), and paclitaxel (TX) in human breast carcinoma cell lines in vitro. In both cell lines, two human breast carcinoma cell lines, MCF-7 and MDA-MB-231, which were tested against DOX, DDP, and TX alone and in combination with ascorbic acid. Ascorbic acid exhibited cytotoxic activity at high concentration (i.e., 10²-10³ μM). Ascorbic acid both at non-cytotoxic (1 μM) and moderately cytotoxic (10² μM) concentration improved the cytotoxicity of DOX, DDP and TX significantly (Kurbacher C M, Wagner U, Kolster B, Andreotti P E, Krebs D, Bruckner H W., Ascorbic acid (vitamin C) cisplatin and paclitaxel in human breast carcinoma cells in vitro., Cancer Lett. Jun. 5, 1996; 103 (2):183-189).

Early studies of vitamin K3 employed an oxidative model to explain the anticancer effects seen in both in vitro and in vivo studies; however, this model does not adequately address the action of vitamins K1 and K2. Recent research has demonstrated that the anticancer action of vitamin K may act at the level of tyrosine kinase and phosphatases, modulating various transcription factors such as Myc and Fos. Tyrosine kinase associated with cyclins have also been shown to be affected by vitamin K, which can lead to cell cycle arrest and cell death. The effect of vitamin K3 on L1210 murine leukemia cell growth has been reported (Chlebowski R T, Dietrich M, Akman S, Block J B. Cancer Treat Rep. May 1985;69(5):527-532).

It is surprisingly found in the invention that a combination of ascorbic acid and vitamin K3 improves the inhibitory effect of chemotherapeutic agents on several carcinoma cell lines. This invention relates to a combination of ascorbic acid and vitamin K3 for augmenting the effectiveness of cancer chemotherapeutics, and to a method for treating tumor/cancer comprising administering the combination to a mammal in need of treatment who is receiving a chemotherapeutic agent(s).

### SUMMARY OF THE INVENTION

It is an object of the invention to provide a pharmaceutical composition for treating tumor/cancer in a mammal in need of treatment comprising a combination of ascorbic acid and vitamin K3 and a chemotherapeutic agent(s) in an effective amount. Preferably, the ascorbic acid is administered at a dose greater than 10 g/day. More preferably, at 30-60 g/day. In addition, the vitamin K3 is administered at a dose greater than 100 mg/day, preferably.

Another object of the invention is to provide a method for treating tumor/cancer comprising administering to a mammal in need of treatment an effective amount of a combination of ascorbic acid and vitamin K3 together with a cancer an anti-tumor amount of a cancer chemotherapeutic agent(s).

Another object of the invention is to provide a method of augmenting the effectiveness of cancer chemotherapeutic agents comprising administering to a mammal who is receiving a chemotherapeutic agent(s) an effective amount of a combination of ascorbic acid and vitamin K3. Preferably, the ascorbic acid is administered at a dose greater than 10 g/day, more preferably at 30-60 g/day. In addition, the vitamin K3 is administered at a dose greater than 100 mg/day, preferably.

A further object of the invention is to provide a therapy combination product for treating tumor/cancer in a mammal comprising in one compartment of a dosage of ascorbic acid, in one compartment of a dosage of vitamin K3 and in another compartment of a dosage of a chemotherapeutic agent.

The details of the invention are set forth in the description below. Other features, objects, and advantages of the invention will be apparent from the description and the claims.

### BRIEF DESCRIPTION OF THE DRAWING

FIG. 1 depicts the effect of a combination of ascorbic acid (1 mM) and vitamin K3 (1 μM) together with As₂O₃ (0.25-2 μg/ml) on the viability of Hep3B cells.

FIG. 2. depicts the effect of a combination of ascorbic acid with varied concentrations (100 μg/ml, 200 μg/ml, 400 μg/ml, 800 μg/ml) and vitamin K3 (1-3 μg/ml) together with As₂O₃ (0.5-2 μg/ml) on the viability of MCF-7 cells.

FIG. 3 depicts the effect of a combination of ascorbic acid with varied concentrations (100-200 μg/ml) and vitamin K3 (0.1 μg/ml) together with 5-FU on the viability of T109A cells.

FIG. 4 depicts the effect of adriamycin at various concentrations (0.03-1 μg/ml) combined with or without a combination of vitamin C (100 or 200 μg/ml) and vitamin K3 (1 μg/ml) on the viability of Hep3B hepatoma cancer cells.

FIG. 5 depicts the effect of cisplatin at various concentrations (0.03-1 μg/ml) combined with or without a combination of vitamin C (100 or 200 μg/ml) and vitamin K3 (1 μg/ml) on the proliferation of Hep3B cancer cells.

### DETAILED DESCRIPTION OF THE INVENTION

The term “anti-tumor amount” as used herein, refers to an amount sufficient to achieve a level of concentration in a mammal's plasma (wherein the mammal is in need of treatment) that is cytotoxic to the cancer cells as demonstrated experimentally using in vitro culture models that mimic the in vivo solid tumor.

The term “effective amount” as used herein, refers to an amount that improves the effectiveness of a chemotherapeutic agent used for treating tumor/cancer in a mammal in need of treatment.

The term “carrier” or “a pharmaceutically acceptable carrier” as used herein, refers to a diluent, an excipient, a recipient and the like for use in preparing admixtures of a pharmaceutical composition.

The term “ascorbic acid,” “ascorbate” or “vitamin C” as used herein, refers to the same meaning for ascorbic acid including the anionic component, ascorbate whether as an acid or one of the pharmaceutically acceptable salt thereof, most notably including sodium ascorbate and calcium ascorbate.

The term “vitamin K3” as used herein, refers to natural or synthetic vitamin K3 and the pharmaceutically acceptable salts thereof.

This invention is based on the discovery of an effect of cancer chemotherapeutic agents is significantly augmented by formulating them with a combination of ascorbic acid and vitamin K3. Moreover, the augmenting effect is increased by the increased concentration of ascorbic acid, especially greater than 200 μg/ml. As demonstrated in the results given in Example 1 (discussed below), a combination of ascorbic acid (100 μg/ml-200 μg/ml) and vitamin K3 (0.1 μg/ml-1 μg/ml) was found to significantly agument the chemotherapeutic agent, such as As₂O₃, adriamycin, cisplatin or 5-Fu, to inhibit several carcinoma cell proliferation.

The present invention features the use of the combination of ascorbic acid and vitamin K3 for augmenting the effectiveness of cancer chemotherapeutic agents against carcinoma cells.

Numerous chemotherapeutic agents have been developed for cancer treatment, such as alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Examples of such chemotherapeutic agents to be used in the methods of the invention include, but are not limited to 5-FU, adriamycin, cisplatin or an arsenic-containing compound.

In a preferred embodiment of this invention, the chemotherapeutic agent suitable for the invention includes, not limited to, 5-FU, adriamycin, cisplatin and an arsenic-containing compound. Preferably, the arsenic-containing compound is As₂O₃.

According to this invention, the method and pharmaceutical composition/therapy combination product can be used in the treatment of a tumor or cancer. Preferably, the tumor or cancer is selected from a group consisting of hematological malignancies and solid tumors. Preferably, the solid tumor is selected from a group consisting of breast cancer, colon cancer rectal cancer, liver tumor, ovarian cancer and prostate cancer. More preferably, the solid tumor is selected from a group consisting of hepatocellualr carcinoma, cholangiocarcinoma, fibrolamellar variant of hepatocellular carcinoma, intrahepatic bile duct carcinoma, mixed hepatocellular cholangicocarinoma, undifferentiated hepatocellular carcinoma, hepatoma with tumor invasion to common bile duct, hepatoblastoma, childhood hepatic tumor, and primary hepatocellular carcinoma.

The present invention provides a pharmaceutical composition for treating tumor/cancer, especially advanced solid tumor, in a mammal comprising an anti-tumor amount of a chemotherapeutic agent(s) and a combination of ascorbic acid and vitamin K3, in an effective amount to augment the effectiveness of a chemotherapeutic agent(s) and at least one pharmaceutically acceptable carrier, diluent or excipient. Preferably, the ascorbic acid is administered at a dose greater than 10 g/day, more preferably at 30-60 g/day. In addition, the vitamin K3 is administered at a dose greater than 100 mg/day, preferably.

A preferred pharmaceutical composition comprises an anti-tumor amount of arsenic-containing compound and an effective amount of a combination of ascorbic acid and vitamin K3; more preferably, the arsenic-containing compound is As₂O₃. Another preferred pharmaceutical composition comprises an antitumor amount of 5-FU, cisplatin or adriamycin and an effective amount of a combination of ascorbic acid and vitamin K3.

The pharmaceutical composition of this invention can be formulated by techniques which are well known to one skill in the art into forms suitable for parenteral, oral, or topical administration, such as solution, capsule, dispersion, suspension, and so on. Preferably, the pharmaceutical composition according to the invention is for parenteral administration. More preferably, the pharmaceutical composition according to this invention is formulated into forms suitable for injection, such as powder injection, lyophilization product for injection, emulsion injection, oily injection, liposome injection, etc. In preparing the pharmaceutical compositions of the present invention, various additives can be included as may be appropriate, such as one or more carriers, diluents, excipients, fluidizing agents, binding agents, stabilizers, thickeners, pH adjusting agents and the like.

Suitable carriers, diluents and excipients include starches and derivatives thereof, such as potato starch, corn starch, dextrin and wheat starch and hydroxypropyl starch; sugars, such as lactose, glucose, sucrose, mannitol and sorbitol; celluloses, such as methylcellulose, carboxylmethylcellulose and hydroxypropylcellulose; inorganic compounds, such as sodium chloride, boric acid, calcium sulfate, calcium phosphate and precipitated calcium carbonate; and the like.

Suitable fluidizing agents include magnesium oxide, synthetic aluminum silicate, metasilicic acid, magnesium aluminum oxide, hydrous silicic acid, anhydrous silicic acid, talc, magnesium stearate, kaolin and the like.

Suitable binding agents include polyethylene glycol, polyvinyl pyrrolidine, polyvinyl alcohol, gum arabic, tragacanth, sodium alginate, gelatin, gluten, and the like.

Suitable stabilizers include proteins, such as albumin, protamine, gelatin and globulin; amino acids and salts thereof, and the like.

Suitable thickeners include sucrose, glycerin, methylcellulose, carboxymethylcellulose, and the like.

Suitable pH adjusting agents include hydrochloric acid, sodium hydroxide, phosphates, citrates, carbonates, and the like.

The invention further provides a method for treating tumor/cancer in a mammal comprising the steps of:
administrating to a mammal in need of treatment of an effective amount of a combination of ascorbic acid and vitamin K3 together with an anti-tumor amount of a chemotherapeutic agent(s).

Preferably, the ascorbic acid is administered at a dose greater than 10 g/day. More preferably, the ascorbic acid is administered at 30-60 g/day. In addition, the vitamin K3 is administered at a dose greater than 100 mg/day, preferably.

A preferred method according to the invention embraces treatment of solid tumors. A further preferred method comprises an effective amount of ascorbic acid and vitamin K3 and an anti-tumor amount of adriamycin, cisplatin, an arsenic-containing compound or 5-FU to treat tumor/cancer including breast cancer, liver cancer and colon cancer.

The present invention also provides a method of augmenting the effectiveness of cancer chemotherapeutic agents comprising the steps of:
administrating to a mammal who is receiving a chemotherapeutic(s) of a combination of ascorbic acid and vitamin K3 in an effective amount to augment the effectiveness of the chemotherapeutic(s).

Preferably, the ascorbic acid is administered at a dose greater than 10 g/day, more preferably at 30-60 g/day. In addition, the vitamin K3 is administered at a dose greater than 100 mg/day, preferably.

Persons skilled in the art will readily be able to determine the most suitable route and dose for the conditions to be treated. Preferably, according to the invention, the preferred route for administration is parenteral administration. Dosages will be at the discretion of the attendant physician or veterinarian, and will depend on the nature and state of the condition to be treated, the age and general state of health of the subject to be treated, the route of administration, and any previous treatments that may have been administered. As known to those skilled in the art, the dosage may vary with the individual depending on the age, size and health conditions and the related factors of the individuals.

The present invention also provides a therapy combination product, in a single compartment comprising:

(a) a ready-to-use pharmaceutical preparation suitable for administration, containing ascorbic acid with a dose greater than 10 g;

(b) a-ready-to-use pharmaceutical preparation suitable for administration, containing vitamin K3 with a dose greater than 100 mg; and

(c) a ready-to-use pharmaceutical preparation suitable for administration, containing a chemotherapeutic agent.

The invention will be described in detail by reference to the following non-limiting examples. The following procedures are carried out to demonstrate the effect of a combination of ascorbic acid and vitamin K3 on carcinoma tumor cell viability.

### EXAMPLE 1

Tumor Samples and Cell Lines

The three human cell lines including human hepatoma cell line Hep3B, breast cancer cell line MCF-7 and colon carcinoma tumor cell line T109A were used in this study.

Anti-Cancer Agents

Sodium ascorbate, vitamin K3, 5-Fu, cisplatin and adriamycin were purchased from Sigma Pharmaceutical Co. As₂O₃ (Asadin) was gifted from Taiwan TOYO Pharmaceutical Co. The above agents were diluted by culture medium at various concentrations and were mixed completely and then added into wells for incubation with cancer cells.

Treatment of Cancer Cells

For treatment, cancer cells were cultured at 37° C. and then plated in 96-well plates at a density of 1×10³ cells/well. After overnight cell attachment at 37° C., wells were exposed to various treatments by direct application of anti-cancer agents with or without addition of vitamin C and vitamin K3 for 72 hours.

MTT Assay

After 72 hours incubation, the supernatant was removed and replaced with fresh medium containing MTT (0.5 mg/ml) at 37° C. for four hours, after which the supernatant was removed and was dissolved with DMSO. Cell viability was determined by standard calorimetric techniques with an EIA reader at a test wavelength of 550 nm.

Viability=absorbance of drug treatment/absorbance of without drug treatment×100%.

Statistical Analysis

For data analysis, the mean of the absorbance is plotted against concentration of chemotherapeutic agent for each treatment to obtain a dose-response relationship of augmenting effect.

Results

Augment Effect of a Combination of Ascorbic Acid and Vitamin K3 on the Anti-Cancer Activity of As₂O₃ Against Hep3B Cells

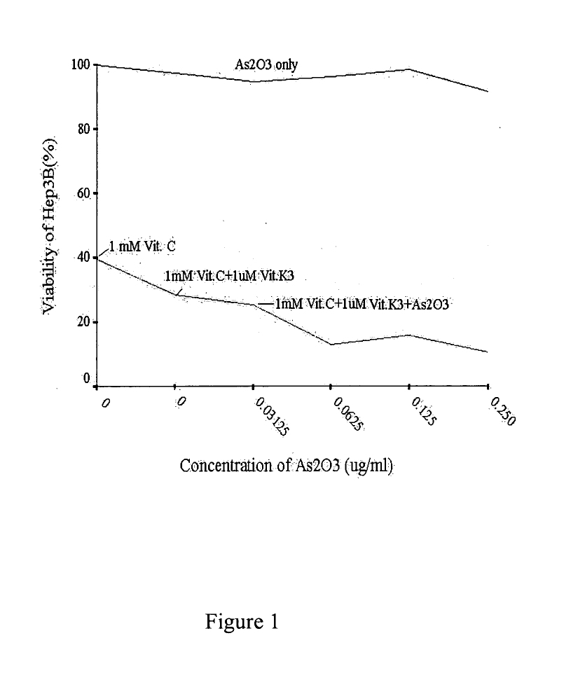

FIG. 1 shows the effect of As₂O₃ alone and As₂O₃ plus a combination of ascorbic acid (1 mM) and vitamin K3 (1 μM) on viability of Hep3B cells. In this experiment, the ascorbic acid and vitamin K3 concentration was kept constant while the As₂O₃ was varied. It is clear from FIG. 1 that combined treatment with the combination of ascorbic acid and vitamin K3 gives greater suppression on Hep3B cell viability than As₂O₃ alone. Note that the augmenting effect on viability inhibition is dose dependent.

Augmenting Effect of a Combination of Ascorbic Acid and Vitamin K3 on the Anti-Cancer Activity of As₂O₃ Against MCF-7 Breast Cancer Cells

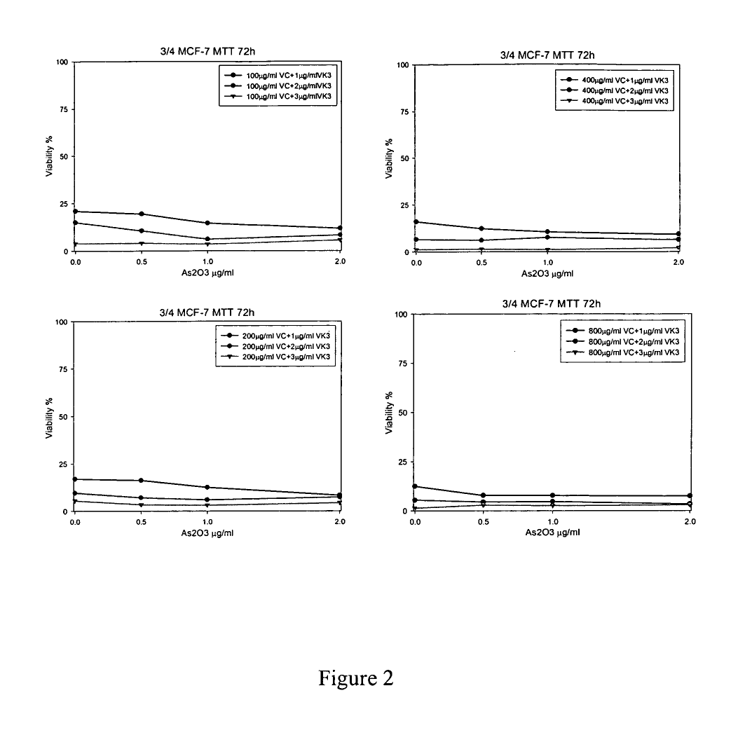

Another experiment was carried out by varying the ascorbic acid and vitamin K3 concentrations while using 100-800 μg/ml of ascorbic acid, 1-3 μg/ml of vitamin K3 and 0.5-2 μg/ml of AS₂O₃. The results shown in FIG. 2 indicate that combined treatment with the combination of ascorbic acid and vitamin K3 gives significant suppression on MCF-7 cell viability. As can be seen from the graphs, the concentration of Vitamin K3 is increased, and the suppression activity is increased.

Augmenting Effect of a Combination of Ascorbic Acid and Vitamin K3 on the Anti-Cancer Activity of 5-FU Against T109A Colon Cancer Cells

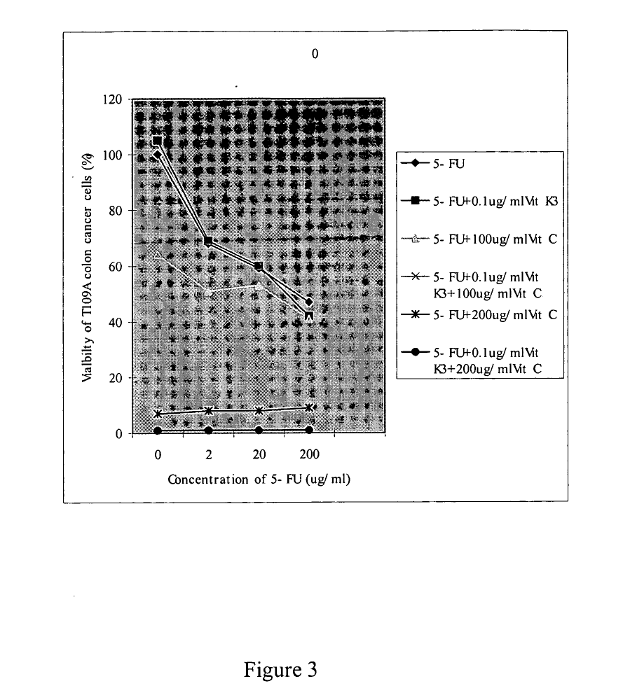

FIG. 3 shows the effect of 5-FU alone, 5-FU plus ascorbic acid, 5-FU plus vitamin K3 and 5-FU plus a combination of ascorbic acid and vitamin K3 on viability of T109A cells. All of 5-FU, the ascorbic acid and vitamin K3 concentration was varied. It is clear from FIG. 3 that combined treatment with the combination of ascorbic acid and vitamin K3 gives greater suppression on T109A cell viability than 5-FU alone/5-FU plus ascorbic acid/5-FU plus vitamin K3. Note that the augment effect on viability inhibition is dose dependent.

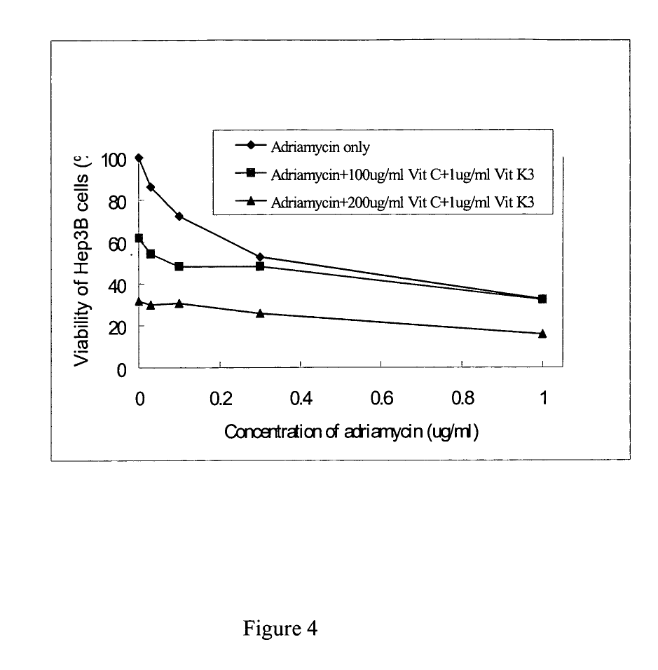

FIG. 4 shows the effect of adriamycin alone, ascorbic acid plus vitamin K3, and adriamycin plus a combination of ascorbic acid and vitamin K3 on viability of Hep3b hepatoma cells. The concentrations of adriamycin were varied between 0.03-1 μg/ml, but the ascorbic acid (100-200 μg/ml) and vitamin K3 (1 μg/ml) were constant. It is clear that combined treatment with the combination of ascorbic acid, vitamin K3 an adriamycin gives greater suppression on Hep3B hepatoma cancer cell viability than adiramycin alone or ascorbic acid plus vitamin K3. This augmenting effect of a combination of vitamin C and vitamin K3 on the anti-cancer effect to adriamycin is more evident when vitamin C is at a high concentration of 200 μg/ml.

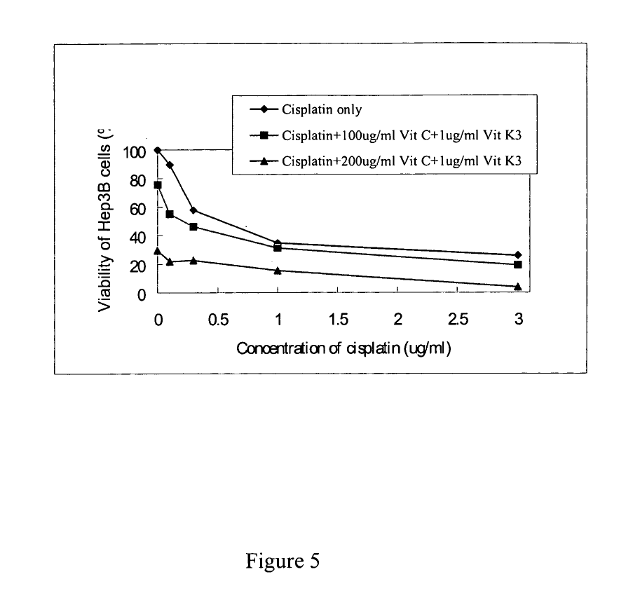

FIG. 5 shows the effect of cisplatin alone, ascorbic acid plus vitamin K3, and cisplatin plus a combination of ascorbic acid and vitamin K3 on viability of Hep3B hepatoma cells. The concentrations of cisplatin were varied between 0.1-3 μg/ml, but the ascorbic acid (100 or 200 μg/ml) and vitamin K3 (1 μg/ml) were constant. It is clear that combined treatment with the combination of ascorbic acid, vitamin K3 and cisplatin gives greater suppression on Hep3B hepatoma cancer cell viability than cisplatin alone or ascorbic acid plus vitamin K3. This augmenting effect of a combination of vitamin C and vitamin K3 on the anti-cancer effect of cisplatin is more evident when vitamin C at a high concentrations of 200 μg/ml.

A combination of ascorbic acid and vitamin K3 indeed improves the viability inhibitory activity of chemotherapeutic agents (e.g., As₂O₃, adriamycin, cisplatin and 5-FU), against hepatoma, breast cancer and colon carcinoma cell lines as evaluated using a viability inhibition assay. The augmenting effect is dose-dependent.

What we claim are:

1. A method for treating tumor/cancer in a mammal comprising the step of: administrating to a mammal in need of treatment an effective amount of a combination of ascorbic acid and vitamin K3 together with an anti-tumor amount of a chemotherapeutic agent(s).

2. The method of claim 1, which is for parenteral or oral administration.

3. The method of claim 1, wherein the ascorbic acid is at a dose greater than 10 g/day.

4. The method of claim 1, wherein the vitamin K3 is at a dose greater than 100 mg/day.

5. The method of claim 1, wherein said chemotherapeutic agent is selected from 5-FU, adriamycin, cisplatin and an arsenic-containing compound.

6. The method of claim 5, wherein the arsenic-containing compound is As₂O₃.

7. The method of claim 1, wherein the tumor/cancer treated is selected from liver cancer, breast cancer or colon cancer.

8. A method of augmenting the effectiveness of cancer chemotherapeutic agents comprising the step of:
administrating to a mammal an effective amount of a combination of ascorbic acid and vitamin K3;
wherein the mammal is receiving a chemotherapeutic agent(s).

9. The method of claim 8, which is for parenteral or oral administration.

10. The method of claim 8, wherein the ascorbic acid is at a dose greater than 10 g/day.

11. The method of claim 8, wherein the vitamin K3 is at a dose greater than 100 mg/day.

12. The method of claim 8, wherein said chemotherapeutic agent is selected from 5-FU, adriamycin, cisplatin and an arsenic-containing compound.

13. The method of claim 12, wherein the arsenic-containing compound is As₂O₃.

14. A pharmaceutical composition for treating tumor/cancer in a mammal comprising an anti-tumor amount of a chemotherapeutic agent(s) and an effective amount of a combination of ascorbic acid and vitamin K3 together with at least one pharmaceutically acceptable carrier, diluent or excipient.

15. The pharmaceutical composition of claim 14, which is for parenteral or oral administration.

16. The pharmaceutical composition of claim 14, wherein the ascorbic acid is at a dose greater than 10 g/day.

17. The pharmaceutical composition of claim 14, wherein the vitamin K3 is at a dose greater than 100 mg/day.

18. The pharmaceutical composition of claim 14, wherein said chemotherapeutic agent is selected from 5-FU, adriamycin, cisplatin and an arsenic-containing compound.

19. The pharmaceutical composition of claim 18, wherein the arsenic-containing compound is As₂O₃.

20. The pharmaceutical composition of claim 14, wherein the tumor/cancer treated is selected from liver cancer, breast cancer and colon cancer.

21. A therapy combination product, in a single compartment comprising:
(a) a-ready-to-use pharmaceutical preparation suitable for administration, containing ascorbic acid;
(b) a-ready-to-use pharmaceutical preparation suitable for administration, containing vitamin K3; and
(c) a-ready-to-use pharmaceutical preparation suitable for administration, containing a chemotherapeutic agent.

22. The product of claim 21, wherein the chemotherapeutic agent is selected from 5-FU, adriamycin, cisplatin and arsenic-containing compound.

23. The product of claim 22, wherein the arsenic-containing compound is As₂O₃.

24. The product of claim 21, which is used for liver cancer, breast cancer or colon cancer treatment.
